(19) United States
(12) Patent Application Publication
(10) Pub. No.: US 2010/0021971 A1
(43) Pub. Date: Jan. 28, 2010

(54) METHOD TO REMOVE REPETITIVE SEQUENCES FROM HUMAN DNA

(75) Inventors: Zhong Chen, Sandy, UT (US); Ning Liu, Beijing (CN)

Correspondence Address: WEILI CHENG, CLAYTON, HOWARTH & CANNON, P.C., P.O.BOX 1909, SANDY, UT, 84091, US

(73) Assignee: CMED TECHNOLOGIES LTD., Road Town, Tortola (VG)

(21) Appl. No.: 12/441,525

(22) PCT Filed: Aug. 16, 2007
(86) PCT No.: PCT/US07/76104

§ 371 Date: Mar. 16, 2009

Related U.S. Application Data
(60) Provisional application No. 60/826,425, filed on Sep. 21, 2006.

(51) Int. Cl. C12P 19/34

(52) U.S. Cl. 435/91.2; 435/91.1

(57) ABSTRACT

The invention discloses an innovative method to deplete repetitive sequences from human DNA. The method comprises (a) providing a source DNA containing both unique and repetitive sequences and sonicating the source DNA to smaller fragments; (b) providing a driver DNA containing sequences complementary to the repetitive sequences of the source DNA and labeled with a non-radioactive label, (c) hybridizing the source DNA and the driver DNA in the presence of a molecule that binds the label to form a complex; (d) removing the hybridized repetitive sequences from the complex by using RNAase and electrophoresis or by incubating with a mixture of phenol, chloroform, and ethanol; and (e) recovering the remaining source DNA wherein said repetitive sequences being significantly removed.

### CROSS-REFERENCE TO RELATED APPLICATIONS

This invention claims priority, under 35 U.S.C. § 120, to the U.S. Provisional Patent Application No. 60/826,425 filed on Sep. 21, 2006, which is incorporated by reference herein.

### TECHNICAL FIELD

The present invention relates to molecular cytogenetics. More specifically, the present invention provides an innovative approach to significantly remove repetitive sequences from human DNA.

### SUMMARY OF THE INVENTION

The invention provides an innovative method to remove repetitive sequences from human DNA. The method comprises (a) providing a source DNA containing both unique and repetitive sequences and sonicating the source DNA to smaller fragments; (b) providing a driver DNA containing sequences complementary to the repetitive sequences of the source DNA and labeled with a non-radioactive label, (c) hybridizing the source DNA and the driver DNA in the presence of a molecule that binds the label to form a complex; (d) removing the hybridized repetitive sequences from the complex by using RNAase and electrophoresis or by incubating with a mixture of phenol, chloroform, and ethanol; and (e) recovering the remaining source DNA wherein said repetitive sequences being significantly removed.

In a preferred embodiment, the driver DNA has a hapten, a biotin or digoxigenin (dig)-labeled repetitive sequences. After the reaction has been completed, the hybridized repetitive sequences are removed by incubating the product of step (c) with, for example, avidin or anti-dig and subtracting the hybridized repetitive sequences by using RNAase and electrophoresis or with phenol, chloroform, and ethanol. The addition of the salt of a weak acid, i.e., sodium acetate, improves the separation. The final repetitive sequences-removed source DNA is recovered as a precipitate by PCR amplification with universal primers.

### DISCLOSURE OF THE INVENTION

Cytogenetics is the microscopic examination of chromosomes arrested during the metaphase stage of cell division. In the early 1960s, chromosome analysis was limited to counting chromosomes, roughly identifying them by size and centromere location, and assigning them to groups A through G. A few syndromes were recognized during this period; for example, three copies of a G group chromosome were identified in individuals with Down syndrome.

In 1970, banding techniques were introduced to the field. This approach used chemical or enzyme treatments to produce characteristic light and dark patterns, called bands, along the arms of the chromosomes. Each of the 46 chromosomes could then be individually identified. Comparisons of bands between homologs, or members of a pair of chromosomes, allows identification of some rearrangements, including translocations, deletions or duplications. The extra chromosome in Down syndrome has been identified as number 21, establishing a genotype-phenotype correlation.

As new culturing techniques were developed, longer chromosomes in prophase or early metaphase with more band resolution were obtained, allowing for “high resolution analysis”. Subtle chromosome abnormalities were observed using these techniques and characterization of syndromes associated with chromosome deletions, such as Prader-Willi and Miller-Dieker, began. Specialized media and manipulation of culturing cells with chemicals such as bromodeoxyuridine (BrdU) have permitted the identification of other chromosome abnormalities. Analyses for the exchange of material between chromatids (sister chromatid exchange studies) are useful in the diagnosis of chromosome breakage syndromes. DNA replication studies allow identification of the inactive X-chromosome by differential staining.

Most recently, molecular cytogenetics, such as fluorescence in situ hybridization (FISH), has expanded the potential of this field. It capitalizes on the accuracy and detail of molecular studies, combined with the well-established techniques of cytogenetics, to gain a deeper understanding of chromosome structure. DNA probes for specific loci or genes on the chromosomes are used in FISH. FISH is a technique that allows DNA sequences to be detected on metaphase chromosomes and interphase nuclei in tissue sections by using DNA probes specific for entire chromosomes or single unique sequences/genes. This method helps confirm structural chromosome changes and identify markers. In general, a specimen is treated with heat and formamide to denature the double-stranded DNA to become single stranded. The target DNA is then available for binding to a DNA probe with a complementary sequence that is also similarly denatured and single stranded. The probe and target DNA then hybridize to each other in a duplex based on complementary base pairing. The probe DNA is labeled directly or indirectly with a fluorescent dye. Hybridization signals on a target material can be visualized through the use of a fluorescence microscope. In comparison with traditional cytogenetics, FISH is much easier to perform and requires much less training for technical personnel involved, and is suitable for widespread utilizations.

Molecular cytogenetic studies, including FISH and other related analyses, are more in demand today than ever before, and many different tissue types are useful for these analyses. Peripheral blood is examined for patients with multiple congenital anomalies and/or mental retardation, infertility, or delayed puberty. Amniotic fluid and chorionic villus sampling are obtained for prenatal chromosome analysis with such indications as advanced maternal age, previous pregnancies with chromosome abnormalities, parental carrier of a chromosome anomaly and abnormal maternal serum screening results. Skin or tissue samples are useful in evaluating miscarriages in families with multiple spontaneous fetal loss. Bone marrow or solid tumor biopsies are examined as an aid in the diagnosis and prognosis of leukemias and cancers.

The following represents a few examples of chromosomal changes in neoplasms. Acute leukemias have been associated with specific chromosomal abnormalities. For example, in pediatric acute lymphocytic leukemia (ALL), those which are hyperdiploid (have more than 46 chromosomes) or the t(12;21) have a favorable prognosis; tetraploid, hypodiploid and those with certain translocations [e.g., t(9;22), t (4; 11)] are found to have a poor prognosis. Several chromosomal findings in acute non-lymphocytic leukemias (ANLL) may be associated with a better prognosis [e.g., inv(16), t(8; 21)]. At times, the chromosomal findings are helpful in the subclassification of leukemia through identification of specific translocations.

However, regular DNA FISH probes have a major problem in hybridization because repetitive sequences contained in chromosomal DNA can cause background signals and cross-hybridizations. Human genomic DNA contains many different types of repetitive sequences. Some of these sequences such as the short highly repetitive sequences Alu and the long repetitive sequences Li, appear in genomic DNA approximately a few kilo-bases apart. One solution is to block these repetitive sequences during hybridization by using commercially available human Cot-1 DNA that contains several different repetitive sequences which is applied to pre-hybridization solution containing a DNA probe with repetitive sequences. Another solution is to directly remove repetitive sequences from DNA probes.

### REFERENCES

Gardner R J M, Sutherland G R: Chromosome Abnormalities and Genetic Counseling. 2nd edition. New York, Oxford University Press, 1996, pp 59-203.
Gersen S L, Keagle M B: The Principles of Clinical Cytogenetics. Totowa, Humana Press, 1999, pp 1-31.
Sandberg A A, Chen Z: Cancer cytogenetics and molecular genetics: Clinical implications. Int J Oncol 7:1241-1251, 1995.
Thompson M W, Mcinnes R R, Willard H F: Thompson and Thompson Genetics in Medicine. 5^{th} ed. Philadelphia, W. B. Saunders, 1991, pp 201-228.
Sandberg A A, Chen Z (2000): FISH Analysis. In: Methods in Molecular Medicine, Vol. 55: Hematologic Malignancies: Methods and Techniques. Faguet G B, eds. Humana Press, Inc.
Chen Z, John Carey (2002): Human Cytogenetics: The Chromosome Basis of Human Diseases. In: Rudolph's Pediatrics. Rudolph C D, Rudolph A M, Hostetter M K, Lister G, Siegel N J, eds. 21^{st} Edition, McGraw-Hill

### MODES FOR CARRYING OUT THE INVENTION

Before the present innovative method to deplete repetitive sequences from human DNA is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof

It must be noted that, as used in this specification and the appended claims, the singular forms “a,” “an,” and “the” include plural referents unless the context clearly dictates otherwise. Thus, for example, reference “a DNA sequence” includes reference to two or more such sequences.

In describing and claiming the present invention, the following terminology will be used in accordance with the definitions set out below.

The invention, as described and disclosed herein, provides an innovative method to deplete repetitive sequences from human DNA. The methods and compositions described herein provide capability for multiple operations to be performed with the utmost accuracy and efficiency. The removed repetitive sequences DNA of the invention is highly specific.

As used herein, “target molecule” refers to a molecule whose presence and/or abundance is being detected. A target molecule can be a whole organism, cellular organelles, or molecules of the organism, or fragments thereof. Most often, a “target molecule” is a polymeric molecule, chromosomes or chromosomal DNA. In preferred embodiments, a “target molecule”is a DNA, genomic DNA, a natural, synthetic, or recombinant nucleic acid molecule, peptide-nucleic acid hybrid, among others, with at least several kilo-base pairs in sizes. Therefore, RNA, mRNA, or related products are not the target molecules for this invention. A target molecule can be derived from any of a number of sources, including animals, plants, insects, and the like. In certain embodiments, the target molecule is a nucleic acid molecule whose sequence structure, presence or absence can be used for certain medical and forensic detection purposes.

As used herein, “chromosome-specific probe” refers to a combination of detectably labeled polynucleotides that have sequences corresponding to (i.e., essentially the same as) the sequences of DNA from a particular chromosome or sub-chromosomal regions of a particular chromosome (i.e., a chromosome arm). Typically, the chromosome-specific probe is produced by amplification (i.e., using the polymerase chain reaction) of the corresponding chromosomal DNA. A chromosome-specific probe of the invention hybridizes in an essentially uniform pattern along the chromosome or sub-chromosomal region from which it is derived.

As used herein, “chromosomal aberration” or “chromosome abnormality” refers to a deviation between the structure of the subject chromosome or karyotype and a normal (i.e., “non-aberrant”) homologous chromosome or karyotype. The terms “normal” or “non-aberrant,” when referring to chromosomes or karyotypes, refers to the predominate karyotype or banding pattern found in healthy individuals of a particular species and gender. Chromosome abnormalities can be numerical or structural in nature, and include aneuploidy, polyploidy, inversions, translocations, deletions, duplications, and the like. Chromosome abnormalities may be correlated with the presence of a pathological condition and a wide variety of unbalanced chromosomal rearrangements leading to dysmorphology with a predisposition to developing a pathological condition.

As used herein, the term “label” includes molecules that are attached to a nucleic acid molecule of the invention and either alone or in combination with a binding partner assisting in the extraction of the repetitive sequences, and/or detection of a hybridization product after hybridization between two nucleic acid molecules of the invention. Most often the label of the invention is a protein-based label, such as biotin, that assists in the extraction of the repetitive sequences with solutions that dissolve and remove proteins.

As used herein, the term “molecules attaching a label” refers to molecules that specifically bind to a label molecule and include, for example, any haptenic or antigenic compound such as digoxigenin and anti-digoxigenin; mouse immunoglobulin and goat anti-mouse immunoglobulin, as well as non-immunological binding pairs such as, for example, biotin-avidin, biotin-streptavidin, hormone-hormone receptors, IgG-protein A, and the like.

As used herein, “nucleic acid molecule” includes genomic DNA, synthetic forms, and mixed polymers, and may be chemically or biochemically modified to contain non-natural or derivatized, synthetic, or semi-synthetic nucleotide bases. Also, included within the scope of the invention are alterations of a wild type or synthetic gene, including, but not limited to, deletion, insertion, substitution of one or more nucleotides, or fusion to other polynucleotide sequences, provided that such changes in the primary sequence of the gene do not alter the ability of the nucleic acid molecule to hybridize with the nucleic acid molecule of interest.

### Source DNA

The source DNA is derived from a variety of sources including non-commercial or commercial nucleic acid libraries, including genomic DNA libraries, for example libraries originated from flow-cytometry sorted human chromosomes and cloned DNA fragments, artificial chromosome libraries, among others. The human chromosome libraries include, for example, chromosomes 1-22 and X/Y, libraries, or a combination thereof. Other libraries include those commercially available under, for example, BD Biosciences Clontech Libraries, Biocompare Genomic Libraries, Stratagen Human Lambda Genomic Libraries, ATCC Genomic Libraries, among others. The source DNA is appropriately sonicated into smaller sizes before subtraction.

### Driver DNA

The invention, as described and disclosed herein, encompasses the use of a driver DNA that hybridizes to the source DNA and thereby acts to remove hybridization signals from ubiquitous repetitive sequences of the source DNA. The driver DNA is selected according to the target nucleic acid molecule being analyzed. For the analysis of human chromosomes, the driver DNA is, for example, Cot-1, or some human DNA fragments that contain predominantly repetitive sequences. The driver DNA is available from a variety of sources such as, for example, human genomic DNA from placenta or white blood cells.

### Label Moieties

Labeled moieties are used in the process of labeling the driver DNA. The driver DNA is labeled with one or more detectable labels to produce detectably labeled molecules and/or products.

Several factors govern the choice of labeling moieties including the effect of the label on the rate of hybridization and binding of the nucleic acid fragments to the target DNA, the accessibility of the bound probe to labeling moieties applied after initial hybridization, the mutual compatibility of the labeling moieties, the nature and intensity of the signal generated by the label, the ease of identification and isolation of labeled products, and the like.

Examples of these labels include any haptenic or antigenic compound in combination with an antibody (i.e., digoxigenin and anti-digoxigenin; mouse immunoglobulin and goat anti-mouse immunoglobulin) as well as non-immunological binding pairs (i.e., biotin-avidin, biotin-streptavidin, hormone-hormone receptors, IgG-protein A, and the like).

A more preferred labeling moiety of the invention is a biotin-avidin or digoxigenin-anti-digoxigenin complex that allows separation and isolation of the labeled molecules via protein separation, as well as enzymatic and magnetic separation techniques (i.e., magnetic beads such as Dynabeads™; fluorescent dyes). Biotin is particularly useful for several reasons, including the high affinity of avidin and streptavidin for biotin, and the high signal amplification because a large number of biotin molecules can be conjugated to a nucleic acid molecule. The biotinilated source and/or carrier nucleic acid molecules form a biotinilated product that is extracted by protein denaturing or protein removing solutions such as phenol/chloroform.

### Universal Primers

Also encompassed within the scope of the invention is the use of universal primers for recovering, through PCR, the repetitive sequences-removed source DNA. Universal primers contain a random hexamer, which may amplify any existing DNA.

This invention is further illustrated by the following examples, which are provided by way of illustration only and are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. Those of skill will readily recognize a variety of non-critical parameters, which are changed or modified to yield essentially similar results.

### EXAMPLE 1

### PCR Amplification And Sonication of Source DNA and Preparation of Biotin Labeled Driver DNA

The degenerate primer, i.e. universal primer, is first used to amplify the source DNA by PCR methods. For instance, 2-3 μl of each selected DNA is added to a PCR reaction mix (50 μl) which contains 10 mM Tris-HCl, pH 8.4, 2 mM MgC2, 50 mM KCl, 200 μM each dNTP, 2 μM primer and 2 units Taq DNA polymerase. The reaction is heated to 96° C. for 2 min, followed by 25 cycles at 94° C. for 1 min, 1 min at 56° C., and 1 min at 72° C., with a 5-min final extension at 72° C. The amplified source DNA is subsequently sonicated at height 0, amplitude 100, for one minute, with ten second intervals submerged in an inverted cup horn sonicator

The driver DNA includes any human genomic DNA that predominantly contains repetitive sequences and is biotin-labeled. The mixture of driver DNA can be labeled with biotin by nick translation. For example, 4-6 μl of 10×dNTPs including biotin-16-dUTP are mixed with 2-4 μg driver DNA and 5 μl DNA Polymerase I/DNase I in a total volume of 50 μl, then incubated at 16° C. for 6 hours.

### EXAMPLE 2

### Hybridizing Driver DNA to Source DNA

Driver DNA (8-10 μg) is labeled with biotin by nick translation. After amplification with the universal primer, 80-100 ng of source DNA is hybridized with 8-10 μg biotin-labeled driver DNA, in 20 μl hybridization solution (6×SSC, 0.2% SDS) at 55° C. overnight. After hybridization, 20 μl Avidin (5 μg/ml)(Vector Laboratories, Inc., Calif.) is added to the hybridization mix and incubated at 37° C. for 20 min.

### EXAMPLE 3

### Removing Repetitive Sequences From Source DNA

Approach one: 5 μl of RNAase (5 mg/ml) are added to the hybridization mixture that is incubated at 37° C. for 30 minutes. Subsequently, DNA in the hybridization mixture is separated by using electrophoresis on a 1% agarose gel. The smaller sized DNA (i.e. the repetitive sequences depleted source DNA) on electrophoresis is collected in TE buffer

Approach two: alternatively, the repetitive sequences can be subtracted from the hybridization mixture with the following procedure: 220-260 μl of ddH2O and 300 μl of buffer saturated phenol are added to the hybridization mixture, vortexed for 30 sec, and centrifuged at 14,000 rpm for 5 min; the supernatant is transferred to a clean tube with 300 μl of phenol:chloroform:isoamyl alcohol (25:24:1), vortexed for 30 sec, and centrifuged at 14,000 rpm for 5 min; the supernatant is transferred to a clean tube with chloroform, vortexed for 30 sec and centrifuged at 14,000 rpm for 5 min again; the supernatant is then transferred to a clean tube and 1/10 volume ratio of 3M Sodium Acetate and 2.5 volume ratio of 100% EtOH are added, mixed and precipitated at −200C overnight; the tube is centrifuged at 14,000 rpm for 30 min, the supernatant is discarded, and the pellet air dried, and re-suspended in 10 μl of dH2O or TE buffer.

### EXAMPLE 4

### Recovery of Precipitated Product by PCR

Universal primers are used to recover the remaining source DNA (i.e., the precipitated product) post the above removing step in EXAMPLE 3. The reaction is cycled 5 times at 94° C. for 1 min, 50° C. for 1 min, and 72° C. for 1 min and then 24 cycles at 94° C. for 1 min, 60° C. for 1 min, and 72° C. for 1 min, with the final extension at 72° C. for 5 min.

### EXAMPLE 5

### Efficiency of the Subtraction Procedures

To determine if the above subtraction procedures efficiently remove avidin-biotin complexes, the following experiment is performed:

The source DNA before and after subtraction is electrophoresed on a 1% agarose gel. Before subtraction, a smear of 600-2000 bp DNA can be observed post sonication. The precipitated product after the subtraction is compared to the sample before subtraction. Removed repetitive sequences or substantially removed repetitive sequences refer to at least about 60% removed repetitive sequences. Our approach has consistently demonstrated that the method of the present invention is efficient to remove the avidin-biotin or anti-dig-dig complexes, thus leading to significant depletion of repetitive sequences from the source DNA.

It is to be understood that the above-described embodiments are only illustrative of application of the principles of the present invention. Numerous modifications and alternative embodiments can be derived without departing from the spirit and scope of the present invention and the appended claims are intended to cover such modifications and arrangements. Thus, while the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

We claim:

1. A method for removing repetitive sequences from a source DNA comprising:
(a) providing a source DNA containing both unique and repetitive sequences;
(b) sonicating the source DNA to smaller DNA fragments;
(c) providing a driver DNA which is complementary to the repetitive sequences in the source DNA and is labeled with a non-radioactive label;
(d) hybridizing the source DNA and the driver DNA in the presence of a molecule that binds the label;
(e) removing the hybridized repetitive sequences of the step (d) by using RNAase followed by electrophoresis or by treatment with phenol and chloroform; and
(f) recovering the remaining source DNA wherein the repetitive sequences being significantly removed.

2. The method of claim 1, wherein the recovering step (f) is performed by PCR with an universal primer.

3. The method of claim 1, wherein the label is a hapten, biotin or digoxigenin and the molecule that binds the label is avidin or anti-digoxigenin respectively.

4. The method of claim 1, said treatment with phenol and chloroform is followed by, addition of acetate and alcohol to form a precipitate.

5. The method of claim 1, wherein the source DNA comprises any human DNA with at least several kilo-base pairs in sizes.

6. The method of claim 1, wherein the labeling process is performed with nick translation, random priming, or PCR methods.

7. The method of claim 2, wherein the general primer contains a random hexamer and is capable of amplifying general DNA.
